(19) United States
(12) Patent Application Publication
(10) Pub. No.: US 2019/0365539 A1
(43) Pub. Date: Dec. 5, 2019

(54) REVERSE VENTRICULAR REMODELING AND PAPILLARY MUSCLE APPROXIMATION

(71) Applicant: Edwards Lifesciences Corporation, Irvine, CA (US)

(72) Inventors: Glen T. Rabito, Lake Forest, CA (US); Alison S. Curtis, Costa Mesa, CA (US); Emil Karapetian, Huntington Beach, CA (US)

(21) Appl. No.: 16/415,998

(22) Filed: May 17, 2019

Related U.S. Application Data
(60) Provisional application No. 62/677,297, filed on May 29, 2018.

(51) Int. Cl. A61F 2/24; A61B 17/04

(52) U.S. Cl. CPC A61F 2/2487; A61F 2/2439; A61F 2220/0075; A61F 2002/249; A61B 2017/0409; A61B 17/0401

(57) ABSTRACT

A cardiac tissue repositioning device comprises a first attachment member configured to be anchored to a first portion of cardiac tissue. The device further comprises a second attachment member configured to be anchored to a second portion of cardiac tissue. The device further comprises an adjustable body configured to be moveable between multiple positions and a locking mechanism configured to control movement of the adjustable body between the multiple positions.

### RELATED APPLICATION

This application claims priority to U.S. Provisional Application No. 62/677,297, filed on May 29, 2018, entitled REVERSE VENTRICULAR REMODELING AND PAPILLARY MUSCLE APPROXIMATION, the disclosure of which is hereby incorporated by reference in its entirety.

### BACKGROUND

### Field

The present disclosure generally relates to the field of valve correction.

### Description of Related Art

Heart valve dysfunction can result in regurgitation and other complications due to valve prolapse from failure of valve leaflets to properly coapt. For atrioventricular valves, papillary muscle position can affect the ability of valve leaflets to function properly.

### SUMMARY

In some implementations, the present disclosure relates to a cardiac device comprising a first attachment member configured to be anchored to a first portion of cardiac tissue, a second attachment member configured to be anchored to a second portion of cardiac tissue, an adjustable body configured to be moveable between multiple positions, and a locking mechanism configured to control movement of the adjustable body between the multiple positions.

The first portion of cardiac tissue may comprise a first papillary muscle disposed in a ventricle of a heart, and the first papillary muscle may be connected to a first leaflet of an atrioventricular heart valve. The second portion of cardiac tissue may comprise a second papillary muscle disposed in the ventricle of the heart, and the second papillary muscle may be connected to a second leaflet of the atrioventricular heart valve. In some embodiments, the first portion of cardiac tissue comprises a first ventricular wall and the second portion of cardiac tissue comprises a second ventricular wall. The adjustable body may be further configured to naturally assume a first position of the multiple positions providing a first distance between the first attachment member and the second attachment member and the locking mechanism may be configured to lock the adjustable body in a second position of the multiple positions for a finite period of time, the second position providing a second distance between the first attachment member and the second attachment member. The second distance may be greater than the first distance.

In some embodiments, the locking mechanism is at least partially composed of a naturally-dissolving material. The adjustable body may be configured to reposition the first portion of cardiac tissue and the second portion of cardiac tissue after the locking mechanism dissolves. In some embodiments, the locking mechanism comprises a spacer disposed between portions of the adjustable body. The locking mechanism may comprise a line configured to fit into an aperture in the adjustable body. In some embodiments, the adjustable body comprises an accordion structure and the adjustable body is configured to naturally assume a collapsed configuration of the accordion structure. The adjustable body may comprise a first elongate arm, a second elongate arm, a first connecting arm extending from the first elongate arm, and a second connecting arm extending from the second elongate arm. The locking mechanism may be configured to couple to the first connecting arm and the second connecting arm at a connection point.

The adjustable body may comprise a spring and a plurality of arm members configured to hold the spring in an at least partially expanded state. In some embodiments, the plurality of arm members comprises two or more telescoping arms. One of the two or more telescoping arms may be configured to be nestingly fit within another of the two or more telescoping arms and the locking mechanism may be configured to hold the two or more telescoping arms in an extended position for a finite period of time. The plurality of arm members may comprise two or more longitudinally overlapping arms. In some embodiments, the first attachment member and the second attachment member are configured to cause formation of fibrotic tissue at the first portion of cardiac tissue and second portion of cardiac tissue, respectively.

In some implementations, the present disclosure relates to a method for anchoring into biological tissue, said method comprising delivering a cardiac device into a ventricle of a heart using a delivery system comprising a catheter. The cardiac device comprises an adjustable body configured to be moveable between multiple positions and a locking mechanism configured to control movement of the adjustable body between the multiple positions. The method further comprises fixing the cardiac device to a first portion of cardiac tissue and a second portion of cardiac tissue of the ventricle.

The adjustable body may further comprise a first attachment member configured to be attached to the first portion of cardiac tissue and a second attachment member configured to be attached to the second portion of cardiac tissue. The adjustable body may be further configured to naturally assume a first position of the multiple positions providing a first distance between the first attachment member and the second attachment member and the locking mechanism may be configured to lock the adjustable body in a second position of the multiple positions for a finite period of time, the second position providing a second distance between the first attachment member and the second attachment member. The second distance may be greater than the first distance.

In some embodiments, the locking mechanism is at least partially composed of a naturally-dissolving material. The cardiac device may be configured to reposition the first portion of cardiac tissue and the second portion of cardiac tissue after the locking mechanism dissolves. In some embodiments, the method further comprises removing the locking mechanism after fibrotic tissue forms around at least a portion of the cardiac device. The adjustable body may comprise an accordion structure and the adjustable body may be configured to naturally assume a collapsed configuration of the accordion structure. In some embodiments, the adjustable body comprises a first elongate arm, a second elongate arm, a first connecting arm extending from the first elongate arm, and a second connecting arm extending from the second elongate arm. The locking mechanism may be configured to couple to the first connecting arm and the second connecting arm at a connection point.

The adjustable body may comprise a spring and a plurality of arm members configured to hold the spring in an at least partially expanded state. In some embodiments, the plurality of arm members comprises two or more telescoping arms. A first telescoping arm of the two or more telescoping arms may be configured to be nestingly fit within a second telescoping arm of the two or more telescoping arms. The locking mechanism may be configured to hold the two or more telescoping arms in an extended position for a finite period of time. The plurality of arm members may comprise two or more longitudinally overlapping arms.

In some implementations, the present disclosure relates to a cardiac device comprising a first means for anchoring to a first portion of cardiac tissue, a second means for anchoring to a second portion of cardiac tissue, a tensioning means configured to be moveable between multiple positions, and a locking means configured to control movement of the tensioning means between the multiple positions.

The first portion of cardiac tissue may comprise a first papillary muscle disposed in a ventricle of a heart, the first papillary muscle being connected to a first leaflet of an atrioventricular heart valve. The second portion of cardiac tissue may comprise a second papillary muscle disposed in the ventricle of the heart, the second papillary muscle being connected to a second leaflet of the atrioventricular heart valve. In some embodiments, the first portion of cardiac tissue comprises a first ventricular wall and the second portion of cardiac tissue comprises a second ventricular wall. The tensioning means may be further configured to naturally assume a first position of the multiple positions providing a first distance between the first means for anchoring and the second means for anchoring. The locking means may be configured to lock the tensioning means in a second position of the multiple positions for a finite period of time, the second position providing a second distance between the first means for anchoring and the second means for anchoring. The second distance may be greater than the first distance.

In some embodiments, the locking means is at least partially composed of a naturally-dissolving material. The locking means may comprise a spacer disposed between portions of the tensioning means. The tensioning means may comprise an accordion structure and the tensioning means may be configured to naturally assume a collapsed configuration of the accordion structure. In some embodiments, the tensioning means comprises a first elongate arm, a second elongate arm, a first connecting arm extending from the first elongate arm, and a second connecting arm extending from the second elongate arm. The locking means may be configured to couple to the first connecting arm and the second connecting arm at a connection point. The tensioning means may comprise a spring and a plurality of arm members configured to hold the spring in an at least partially expanded state.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a cross-sectional view of a human heart.

FIG. 2 provides a cross-sectional view of the left ventricle and left atrium of an example heart.

FIG. 3 provides a cross-sectional view of a heart experiencing mitral regurgitation.

FIGS. 4A and 4B illustrate cross-sections of a heart having a tension device disposed therein according to one or more embodiments.

FIGS. 4C and 4D illustrate closer views of the tension device according to one or more embodiments.

FIGS. 5A and 5B illustrate cross-sections of a heart having a pinch device implanted therein according to one or more embodiments.

FIGS. 6A and 6B illustrate extension devices according to one or more embodiments.

FIGS. 6C and 6D illustrate cross-sections of a heart having an extension device and a torsion device implanted therein according to one or more embodiments.

FIG. 7 is a flow diagram illustrating a process for repositioning portions of cardiac tissue according to one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

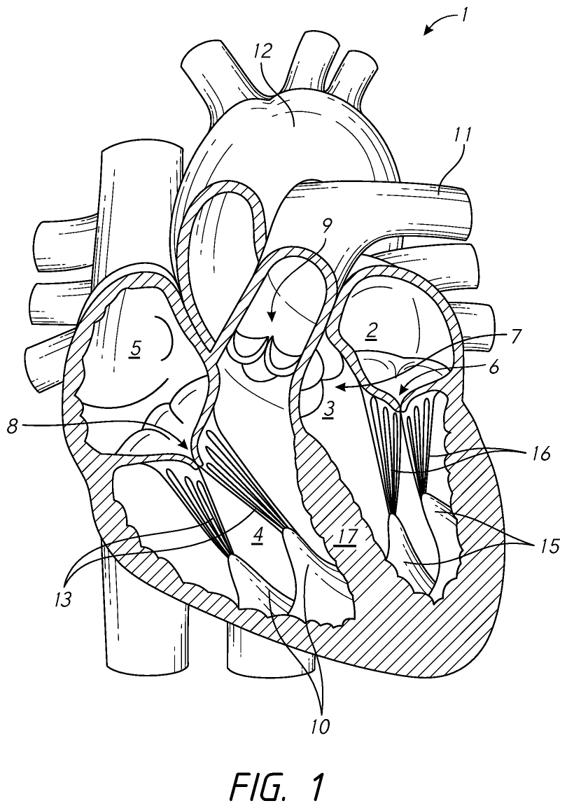

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (i.e., mitral and tricuspid) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles by the chordae tendineae 11, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to the anterior, posterior and septal leaflets of the tricuspid valve, respectively, via the chordae tendineae 11. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

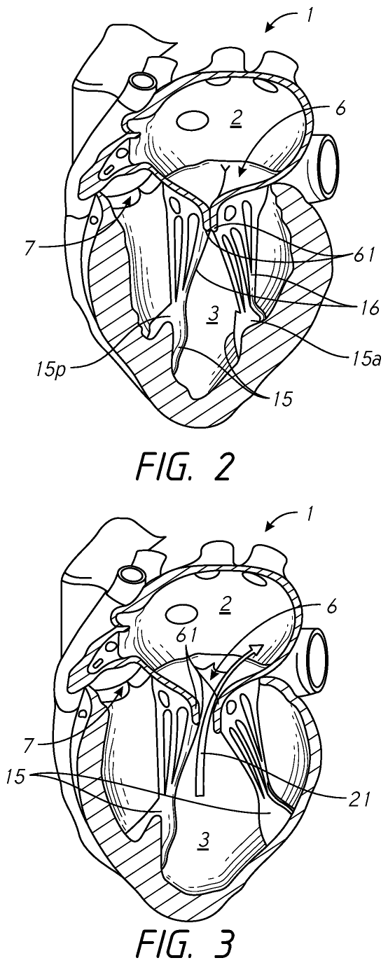

FIG. 2 provides a cross-sectional view of the left ventricle 3 and left atrium 2 of an example heart 1. The diagram of FIG. 2 shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2 and various other figures provided herewith and described herein in the context of certain embodiments of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve as shown in FIG. 2, the valve leaflets 61 may extend inward from the valve annulus and come together in the flow orifice to permit flow in the outflow direction (e.g., the downward direction in FIG. 2) and prevent backflow or regurgitation toward the inflow direction (e.g., the upward direction in FIG. 2). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae and therefore allowing the chordae tendineae to hold the leaflets in a closed position. The papillary muscles 15 may include an anterolateral papillary muscle 15a, which may be tethered to the posterior leaflet, for example, and a posteromedial papillary muscle 15p, which may be tethered to the anterior leaflet, for example. With respect to the state of the heart 1 shown in FIG. 2, the proper coaptation of the valve leaflets, which may be due in part to proper position of the papillary muscles 15, may advantageously result in mitral valve operation substantially free of leakage.

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3, which illustrates a cross-sectional view of a heart 1 experiencing mitral regurgitation flow 21, dilation of the left ventricle may cause changes in the position of the papillary muscles 15 that allow flow 21 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

With further reference to FIG. 3, the heart 1 is shown in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIG. 3 may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3, the leaflets 61 of the mitral valve (or tricuspid valve) are not in a state of coaptation, resulting in an opening between the mitral valve leaflets 61 during the systolic phase of the cardiac cycle, which allows the leakage flow 21 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3) and/or unwanted backflow or regurgitation toward the inflow direction (e.g., the downward direction in FIG. 2).

Certain embodiments disclosed herein provide solutions for incompetent heart valves that involve ventricular wall and/or papillary muscle repositioning. Solutions presented herein may be used to at least partially change the position of one or more papillary muscles and/or ventricular walls in order to reduce the occurrences and/or severity of regurgitation, such as mitral regurgitation. Mitral valve regurgitation often may be driven by the functional/physical positioning changes described above, which may cause papillary muscle displacement and/or dilatation of the valve annulus. As the papillary muscles move away from the valve annulus, the chordae connecting the muscles to the leaflets may become tethered. Such tethering may restrict the leaflets from closing together, either symmetrically or asymmetrically, depending on the relative degree of displacement between the papillary muscles. Moreover, as the annulus dilates in response to chamber enlargement and increased wall stress, increases in annular area and changes in annular shape may increase the degree of valve insufficiency.

Various techniques that suffer from certain drawbacks may be implemented for treating mitral valve dysfunction, including surgical repair or replacement of the diseased valve or medical management of the patient, which may be appropriate/effective primarily in early stages of mitral valve dysfunction, during which levels of regurgitation may be relatively low. For example, such medical management may generally focus on volume reductions, such as diuresis or afterload reducers, such as vasodilators, for example. Valve replacement operations may also be used to treat regurgitation from valve dysfunction. However, such operations can result in ventricular dysfunction or failure following surgery. Further limitations to valve replacement solutions may include the potential need for lifelong therapy with powerful anticoagulants in order to mitigate the thromboembolic potential of prosthetic valve implants. Moreover, in the case of biologically-derived devices, such as those used as mitral valve replacements, the long-term durability may be limited. Another commonly employed repair technique involves the use of annuloplasty rings to improve mitral valve function. An annuloplasty may be placed in the valve annulus and the tissue of the annulus sewn or otherwise secured to the ring. Annuloplasty rings can provide a reduction in the annular circumference and/or an increase in the leaflet coaptation area. However, annuloplasty rings may flatten the saddle-like shape of the valve and/or hinder the natural contraction of the valve annulus. In addition, various surgical techniques may be used to treat valve dysfunction. However, such techniques may suffer from various limitations, such as requiring opening the heart to gain direct access to the valve and the valve annulus. Therefore, cardiopulmonary bypass may be required, which may introduce additional morbidity and mortality to the surgical procedures. Additionally, for surgical procedures, it can be difficult or impossible to evaluate the efficacy of the repair prior to the conclusion of the operation.

Disclosed herein are devices and methods for treating valve dysfunction without the need for cardiopulmonary bypass and without requiring major remodeling of the dysfunctional valve. In particular, passive techniques to lower ventricular volume and/or change the shape and/or position of the papillary muscles are disclosed for improving ventricular function and/or reducing regurgitation while maintaining substantially normal leaflet anatomy. Further, various embodiments disclosed herein provide for the treatment of valve dysfunction that can be executed on a beating heart, thereby allowing for the ability to assess the efficacy of the ventricular remodeling and/or papillary muscle repositioning treatment and potentially implement modification thereto without the need for bypass support.

Some embodiments described herein provide devices and/or methods which involve applying minimal and/or relatively little force to the native tissue at or around the time of implantation, and may apply increased force after a period of time through delayed-loading. Such increase in force may be introduces gradually over time, or in one or more discrete steps. Certain embodiments disclosed herein may provide one or more advantages over other anchoring devices. For example, generally, when certain tissue anchors are fixed or embedded into cardiac tissue (e.g., myocardium and/or endocardium), there may be a substantial risk that the tissue anchor may tear through the cardiac tissue and become dislodged. This may be particularly a concern when the tissue anchor is attached to a load that applies a pulling and/or pushing force to the tissue anchor.

When tissue anchors are placed in cardiac tissue, the tissue anchors may cause trauma and result in the formation of fibrotic scar tissue around the tissue anchors. Fibrotic tissue may be structurally more substantial than the native tissue. Therefore, a tissue anchor may have a lower risk of tearing through a tissue wall after the formation of fibrotic tissue. Accordingly, by delaying loading of tissue anchors for a period of time after insertion, the tissue anchor may be more fixed in the cardiac tissue at the point of loading and may be less likely to become dislodged from the cardiac tissue.

Some embodiments disclosed herein involve delaying loading of tissue anchors through use of locking mechanisms and/or means for locking a repositioning device. The locking mechanisms and/or means for locking may be composed of a naturally-dissolving material and may be configured to hold a repositioning device connected to the tissue anchors in an unloaded position until the locking mechanisms and/or means for locking dissolve. After the locking mechanisms and/or means for locking dissolve, the repositioning device may apply a load to the tissue anchors as a result of the repositioning device relaxing to a natural and/or pre-defined position. In certain embodiments, the repositioning device may comprise a shape memory alloy (e.g., Nitinol) that may cause the repositioning device to move towards a pre-defined position after a period of time. A locking mechanism and/or means for locking may comprise one or more of various objects, including a suture, clip, clasp, hook, rod, pin, tie, net, spacer, cord, or other object or set of objects.

### Tension Device

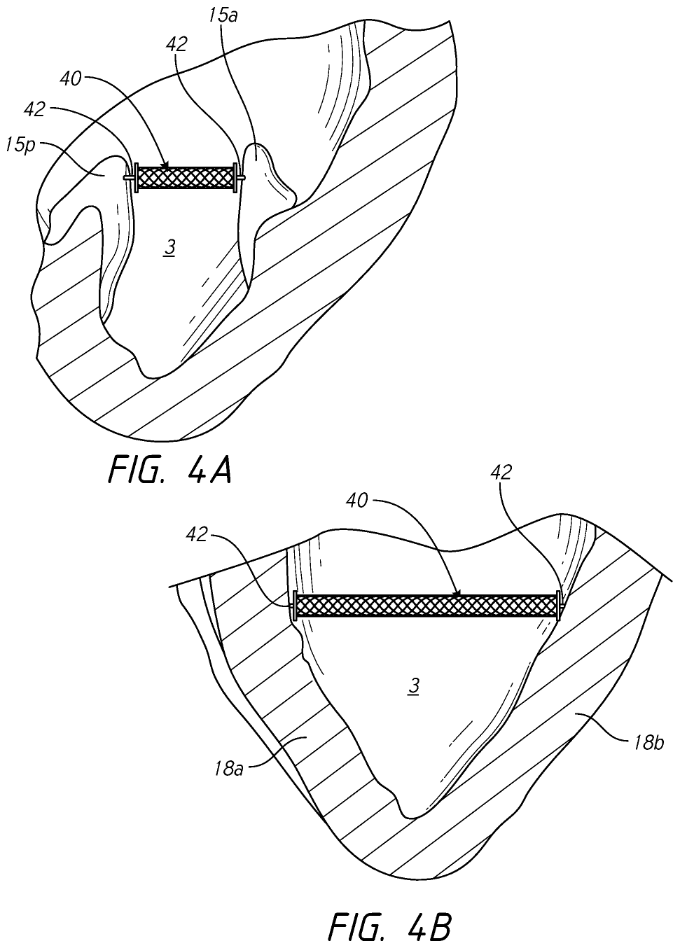

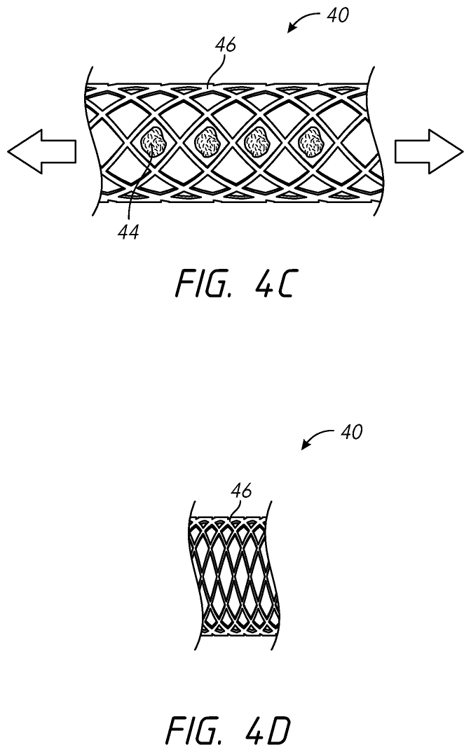

FIGS. 4A and 4B illustrates a cross-section of a heart 1 showing a left ventricle 3 thereof. Although certain disclosure herein is presented in the context of the left ventricle and associated anatomy (e.g., valves, papillary muscles, chordae tendineae, ventricle wall, etc.), it should be understood that the principles disclosed herein may be applicable in any ventricle of the heart (e.g., right ventricle) and associated anatomy (e.g., tricuspid valve, papillary muscles, chordae tendineae, ventricle wall, etc.). As described above, in a normal heart, the papillary muscles may contract during the heart cycle to assist in maintaining proper valve function. Reductions in, or failure of, the papillary muscle function can contribute to valve dysfunction and/or regurgitation, which may be caused by infarction at or near the papillary muscle, ischemia, or other causes, such as idiopathic dilated cardiomyopathy, for example.

FIG. 4A shows a tension device 40, which may be implanted in the left ventricle 3 (or right ventricle in another embodiment) to at least partially pull the posterior-medial papillary muscle 15p and/or the anterolateral papillary muscle 15a towards each other. That is, the tension device 40 may pull the posterior-medial papillary muscle 15p towards the anterolateral papillary muscle 15a and/or the tension device 40 may pull the anterolateral papillary muscle 15a towards the posterior-medial papillary muscle 15p, which may cause one or both of the papillary muscles to reposition inward. By repositioning the papillary muscles inward, the traction of the chordae tendineae on the corresponding leaflet of the mitral valve may be lessened, thereby resulting in improved coaptation of the mitral valve leaflets during closure of the valve. In certain conditions/patients, moving the posterior-medial papillary muscle 15p and the anterolateral papillary muscle 15a closer together may help correct mitral valve insufficiency due to dysfunction or rupture of the papillary muscles.

FIG. 4B shows the tension device 40 anchored to a first ventricular wall 18a and a second ventricular wall 18b. The tension device 40 may be configured to pull the ventricular walls together, thereby lowering ventricular volume. In certain embodiments, the tension device 40 may be anchored to three or more ventricular walls and/or may be anchored to more than two portions of ventricular walls.

The tension device 40 may be anchored to the papillary muscles and/or ventricular walls by one or more anchors or attachment members 42. The attachment members 42 may comprise corkscrews, barbs, balloons, hooks, and/or any other anchoring mechanism suitable for anchoring the tension device 40 to a tissue wall. In some embodiments, the tension device 40 may comprise more than two attachment members 42. For example, because some ventricles may contain three papillary muscles, the tension device 40 may comprise three or more attachment members 42, with at least one attachment member 42 anchored to each papillary muscle. Moreover, multiple attachment members 42 may be anchored to a single papillary muscle and/or ventricular wall.

With respect to embodiments in which the tension device 40 is implanted in the right ventricle, the device may serve to correct tricuspid regurgitation, which, similar to mitral regurgitation, involves a disorder in which the tricuspid valve does not close tightly enough to prevent backflow through the valve. During tricuspid regurgitation, blood may flow backward into the right atrium when the right ventricle contracts. Such tricuspid valve dysfunction may result from the increase in size of the right ventricle. For example, enlargement or dilation of the right ventricle may result from high blood pressure in the arteries of the lungs, or from other heart problems, such as poor squeezing of the left side of the heart, or from problems with the opening or closing of another one of the heart valves.

FIGS. 4C and 4D show closer views of respective portions of the tension device 40. The tension device 40 may have an extendable structure and may be adjustable between a stretched position (illustrated in FIG. 4C) and a collapsed position (illustrated in FIG. 4D). In certain embodiments, the tension device 40 may be composed of mesh-like wire structure, an accordion-structure, a spring-type structure, and/or other extendable structure(s). In the example shown in FIGS. 4C and 4D, the tension device 40 may be comprised of wires 46 composed of plastic, metal, Nitinol, polymer, and/or other material.

As shown in FIG. 4C, the tension device 40 may comprise one or more spacers 44 situated in cavities, gaps, apertures, or voids of the tension device 40. In some embodiments, the one or more spacers 44 may be connected to the wires 46 of the tension device 40.

The one or more spacers 44 may be configured to lock the tension device 40 in a stretched position or configuration. While FIG. 4C shows four spacers 44, there may be fewer or more spacers 44, as needed to hold the tension device 40 in a desired position. The spacer(s) 44 may be composed of a naturally-dissolving (e.g., biodegradable) material that may be configured to dissolve after a period of time (e.g., several days, weeks, and/or months). At insertion, the tension device 40 may be in the stretched position/configuration due to the presence of the spacers 44. In certain embodiments, the spacers 44 may be balloons and/or similar devices that may be inflated after insertion into the patient's body. After a period of time, the spacers 44 may dissolve and/or may be removed, and the tension device 40 may adjust to a collapsed position or configuration. In certain embodiments, each of the spacers 44 may dissolve at different times, causing a gradual change from the stretched position to the collapsed position, or may dissolve at approximately the same time. In embodiments in which the spacers 44 dissolve and/or are removed at different times, the tension device 40 may have one or more intermediary positions between the stretched position shown in FIG. 4C and the collapsed position shown in FIG. 4D.

In the stretched position, the tension device 40 may apply a minimal load to the cardiac tissue. For example, the tension device 40 may be configured to have a natural state in the collapsed position. The spacers 44 may prevent the tension device 40 from collapsing to the natural state. However, when the spacers 44 dissolve, the tension device 40 may apply a pulling force to the papillary muscles and/or ventricular walls as the tension device 40 attempts to move to the collapsed state.

The spacers 44 may be configured to dissolve after a period of time, for example after the formation of at least some fibrotic tissue around the attachment members 42. Thus, the tension device 40 may not apply a load and/or may apply a minimal load to the papillary muscles and/or ventricular walls until the formation of some fibrotic tissue. When the spacers 44 dissolve and/or are removed, the formed fibrotic tissue may provide greater retention for the attachment members 42, which may allow the attachment members 42 to better withstand the pulling force applied by the tension device 40. In this way, a pulling force and/or a relatively greater pulling force may not be applied by the tension device 40 until after the formation of fibrotic tissue.

While in certain embodiments an attachment member 42 may pierce cardiac tissue, an attachment member may alternatively or additionally wrap around or otherwise contact a papillary muscle and/or other cardiac surface or tissue. For example, an attachment member may comprise a cloth that may be configured to wrap around a papillary muscle. The cloth may comprise an open-cell type structure. In response to the contact of the attachment member, fibrotic tissue may form around the cardiac tissue in the area of attachment of the attachment member 42.

A delivery system for the tension device 40 may include a catheter for navigating the tension device 40 to the desired position. For example, the tension device 40 may be delivered to the implantation location in the stretched state (e.g., as shown in FIG. 4C), and may change to the collapsed state (e.g., as shown in FIG. 4D) after a period of time. In other embodiments, the tension device 40 may be delivered to the implantation location in an at least partially collapsed or contracted state, wherein the spacers 44 may be inflated or expanded after being deployed from the delivery catheter. In certain embodiments, the spacers may be inserted in, or attached to, the tension device 40 after deployment from the delivery catheter. The tension device 40 may be inserted non-surgically in, for example, a transcatheter procedure (e.g., transfemoral, transseptal, transapical, etc.), wherein the tension device 40 is inserted into the left ventricle 3 from the aorta 12 through the aortic valve 7 and positioned between papillary muscles and/or ventricular walls. With respect to right ventricle papillary muscle and/or ventricular wall repositioning, the tension device 40 may be inserted into the right ventricle from the pulmonary artery through the pulmonary valve and positioned between the papillary muscles and/or ventricular walls of the right ventricle.

### Pinch Device

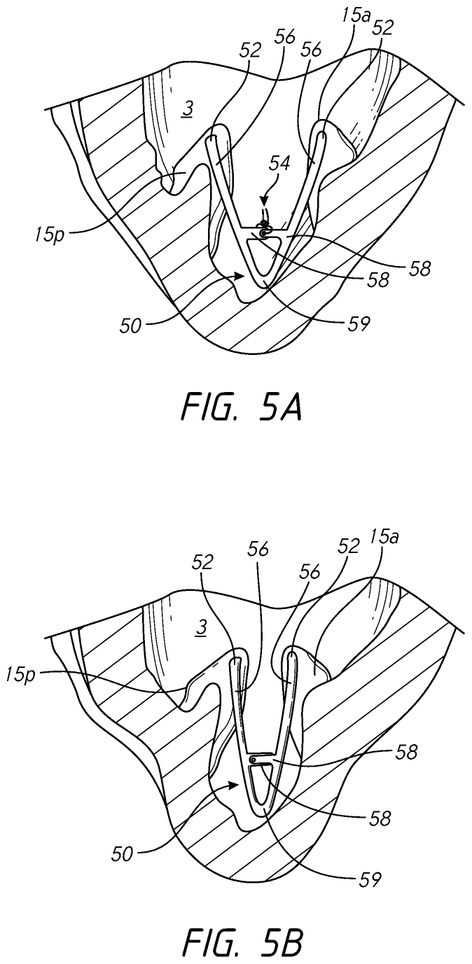

FIGS. 5A and 5B show a pinch device 50 in two different positions in the left ventricle 3 (or right ventricle in another embodiment) anchored between two papillary muscles (e.g., posterior-medial papillary muscle 15p and anterolateral papillary muscle 15a). In certain embodiments, the pinch device 50 may be anchored between ventricular walls. The pinch device 50 may be used independently of or in conjunction with the tension device 40 of FIGS. 4A-4D.

The pinch device 50 may be configured to at least partially pull the posterior-medial papillary muscle 15p and the anterolateral papillary muscle 15a towards each other. That is, the pinch device 50 may pull the posterior-medial papillary muscle 15p towards the anterolateral papillary muscle 15a and/or the pinch device 50 may pull the anterolateral papillary muscle 15a towards the posterior-medial papillary muscle 15p, which may cause the papillary muscles to reposition inward. In certain embodiments, the pinch device 50 may be anchored between ventricular walls and may be configured to apply pulling force to the ventricular walls to reduce ventricular volume.

The pinch device 50 may be anchored to the papillary muscles and/or ventricular walls by one or more anchors or attachment members 52. The attachment members 52 may comprise corkscrews, barbs, balloons, hooks, and/or any other anchoring mechanism suitable for anchoring the pinch device 50 to a tissue wall. In some embodiments, the pinch device 50 may comprise more than two attachment members 52. For example, because some ventricles may contain three papillary muscles, the pinch device 40 may comprise three or more attachment members 52, with at least one attachment member 52 anchored to each papillary muscle. Moreover, multiple attachment members 52 may be anchored to a single papillary muscle and/or ventricular wall. With respect to embodiments in which the pinch device 50 is implanted in the right ventricle, the device may serve to correct tricuspid regurgitation.

FIG. 5A shows an initial and/or first stage of the pinch device 50 and FIG. 5B shows a final and/or second stage of the pinch device 50. The pinch device 50 may have an adjustable structure and may be adjusted between a stretched position (illustrated in FIG. 5A) and a collapsed position (illustrated in FIG. 5B). The pinch device 50 may comprise one or more anchoring arms 56 and/or one or more extension arms 58. The one or more anchoring arms 50 may be configured to anchor to a tissue wall (e.g., posterior-medial papillary muscle 15p and/or anterolateral papillary muscle 15a) using one or more attachment members 52. In certain embodiments, the pinch device 50 may comprise two anchoring arms 56 connected at a joint 59. In some embodiments, the pinch device 50 may comprise three or more anchoring arms 56 connected at the joint 59. The one or more anchoring arms 56 may be composed of a single length of material that may be bent to form the joint 59. The one or more anchoring arms 56, extension arms 58, and/or joint 59 may be composed of plastic, metal, polymer, or other material and may be substantially rigid in form such that the one or more one or more anchoring arms 56, extension arms 58, and/or joint 59 may hold a pre-defined orientation and may be resistant to applied force. For example, if a force is applied to push multiple anchoring arms 56 together and/or pull the anchoring arms 56 apart, the anchoring arms 56 may apply a resistive force and may return to the pre-defined orientation when the force is removed.

In some embodiments, each of the one or more extension arms 58 may extend from an anchoring arm 56. In certain embodiments, the pinch device 50 may comprise multiple extension arms 58, each extending from a different anchoring arm 56. The multiple extension arms 58 may be disconnected from each other or may be slidably connected to each other. For example, a first extension arm 58 may comprise a connection track and a second extension arm 58 may comprise a connection peg that may slidably connect to the connection track such that the connection peg may be slidable between a first end of the connection track and a second end of the connection track. The connection track may cover an entire length or at least a portion of the first extension arm 58. In some embodiments, the extension arms may connect via a prismatic joint, a cylindrical joint, and/or another mechanism, wherein a first extension arm 58 may be hollow and a second extension arm 58 may be sized to nestingly fit into the first extension arm 58. In such embodiments, the first extension arm 58 and/or second extension arm 58 may have a retention mechanism to prevent the second extension arm 58 from becoming disconnected from the first extension arm 58.

The pinch device 50 may be held in a first position (illustrated, e.g., in FIG. 5A) by a naturally-dissolving locking mechanism 54. In certain embodiments, the locking mechanism 54 may comprise a line or suture configured to pass through holes in the one or more extension arms 58 to hold the extension arms 58 in place. The term “line” is used herein according to its broad and ordinary meaning and may refer to a string, cord, wire, or other length of material. In some embodiments, the locking mechanism 54 may be a spacer or any other mechanism suitable for holding the extension arms 58 in place. For example, the locking mechanism 54 may prevent the extension arms 58 from overlapping each other beyond a certain point. In certain embodiments, the locking mechanism 54 may prevent a connection mechanism of the one or more extension arms 52 from sliding or otherwise changing positions. The locking mechanism 54 may be composed of naturally-dissolving (e.g., bio-absorbable) material that may be configured to dissolve after a period of time.

When the locking mechanism 54 dissolves or is removed, the pinch device 50 may change to a second position (illustrated in FIG. 5B). The second position may be a pre-defined and/or natural orientation of the pinch device 50 such that when there is no external force and/or no locking mechanism 54 applied to the pinch device 50, the pinch device 50 may naturally rest in the second position.

As shown in FIGS. 5A and 5B, the extension arms 58 may apply a pushing force against the anchoring arms 56. In the first position (illustrated, e.g., in FIG. 5A), there may be little or no overlap between the extension arms 58. Accordingly, the extension arms 58 may have a greater overall length and may force the anchoring arms 56 further apart. In the second position (illustrated, e.g., in FIG. 5B), there may be greater and/or complete longitudinal overlap between the extension arms 58. Accordingly, the extension arms 58 may have a smaller overall length and may apply little or no pushing force against the anchoring arms 56, resulting the in the anchoring arms 56 relaxing inwards. As the anchoring arms 56 relax inwards (i.e., towards each other), they may apply a pulling force to the papillary muscles and/or ventricular walls to cause the papillary muscles and/or ventricular walls to move closer together. In the first position (illustrated, e.g., in FIG. 5A), the pinch device 50 may apply minimal pulling force to the papillary muscles and/or ventricular walls. The distance between the anchoring arms 56 may be greater in the first position than in the second position.

A delivery system for the pinch device 50 may include a catheter for navigating the pinch device 50 to a desired location within a patient's body. For example, the pinch device 50 may be delivered in the first position and may adjust to the second position after a period of time. The pinch device 50 may be inserted non-surgically in, for example, a transcatheter procedure (e.g., transfemoral, transseptal, transapical, etc.), wherein the pinch device 50 is inserted into the left ventricle 3 from the aorta 12 through the aortic valve 7 and positioned between the papillary muscles and/or ventricular walls. With respect to right ventricle papillary muscle repositioning, the device 20 may be inserted into the right ventricle from the pulmonary artery through the pulmonary valve and positioned between the papillary muscles of the right ventricle.

### Torsion Device

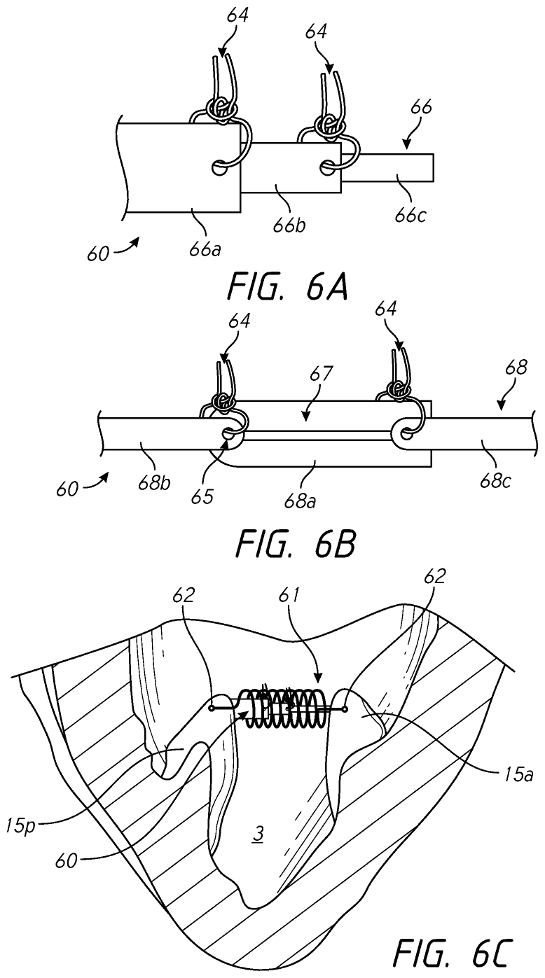

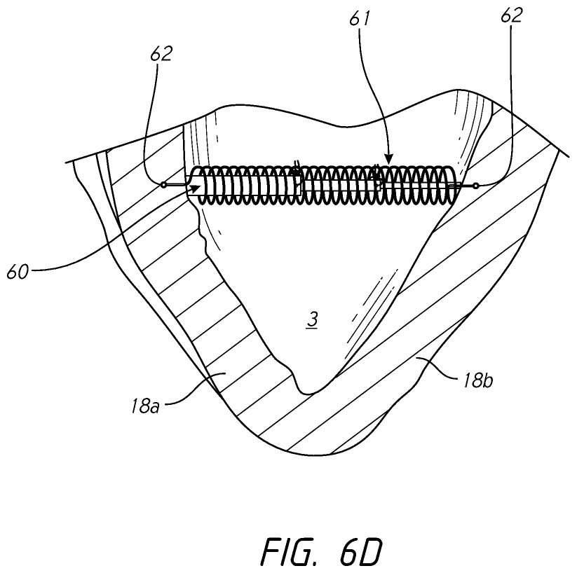

FIGS. 6A and 6B show two variations of an extension device 60 that may be used for ventricular remodeling and/or papillary muscle approximation. As shown in FIG. 6A, the extension device 60 may have a telescoping structure in which multiple arms 66 have variable sizes. The extension device 60 may be used independently of or in conjunction with the tension device 40 of FIGS. 4A-4D and/or the pinch device 50 of FIGS. 5A-5B.

One or more of the arms 66 may have a hollow structure or may otherwise be configured to receive and/or overlap other arms 66. For example, a first arm 66a may be configured to receive a second arm 66b and/or the second arm 66b may be configured to receive a third arm 66c. The arms 66 may be connected or may not be connected. For example, the extension device 60 may comprise a first arm 66a having a hollow cylindrical shape with a first radius and the extension device 60 may also comprise a second arm 66b with a second radius that is smaller than the first radius. In this example, the second arm 66b may be configured to nestingly fit into the first arm 66a. The second arm 66b may also be configured to extend out of the first arm 66a and the first arm 66a and/or second arm 66b may have a retention mechanism to prevent the second arm 66b from disconnecting from the first arm 66a. In another example, a first arm 66a and/or a second arm 66b may have a cubic structure as shown in FIG. 6A. The arms 66 may be composed of plastic, metal, polymer, or other material.

As shown in FIG. 6B, the extension device 60 may have an overlapping structure in which multiple arms 68 may be configured to have a prismatic joint, a cylindrical joint, and/or a slider joint such that the arms 68 may be moveable to create varying amounts of longitudinal overlap between the arms 68. For example, the arms 68 may be configured such that, at a first position, a first arm 68a may be entirely or almost entirely coextensive with a second arm 68b. At other positions, the first arm 68a may be mostly and/or at least partially non-coextensive with the second arm 68b. The first arm 68a and/or the second arm 68b may have a connection track 67 and/or a connection mechanism (e.g., a peg configured to fit into the connection track 67) that allows the arms 68 to move with respect to each other and adjust an amount of longitudinal overlap between the arms 68. For example, a peg may be situated between a cavity 65 and an end of an arm 68b to allow the arm 68b to slide along the connection track 67. The extension device 60 may independently include the telescoping arms 66 of FIG. 6A or the overlapping arms 68 of FIG. 6B or may include both the telescoping arms 66 of FIG. 6A and the overlapping arms 68 of FIG. 6B.

The extension device 60 may be held in a first position (illustrated, e.g., in FIG. 6A and FIG. 6B) by a naturally-dissolving locking mechanism 64. In certain embodiment, the locking mechanism 64 may comprise a line or suture configured to pass through cavities 65 in the arms 66, 68 to hold the arms 66, 68 in place. In some embodiments, the locking mechanism 64 may be a spacer or any other mechanism suitable for holding the arms 66, 68 in place. For example, the locking mechanism 64 may prevent any of the arms 66, 68 from sliding or otherwise changing positions. The locking mechanism 64 may be composed of naturally-dissolving (e.g., bio-absorbable) material that may be configured to dissolve after a period of time.

As shown in FIGS. 6C and 6D, the extension device 60 may be utilized in conjunction with a torsion device 61. In some embodiments, the torsion device 61 may be connected to the extension device 60, but in other embodiments the torsion device 61 may not be connected to the extension device 60. The torsion device 61 may be anchored between papillary muscles (as shown, e.g., in FIG. 6C) and/or ventricular walls (as shown, e.g., in FIG. 6D) via attachment members 62. The attachment members 62 may comprise corkscrews, barbs, balloons, hooks, and/or any other anchoring mechanism suitable for anchoring the torsion device 61 to a tissue wall. The torsion device 61 may be a spring, stent, or other device and may naturally rest in a collapsed position. While FIGS. 6C and 6D show two attachment members 62, the torsion device 61 may comprise fewer or more attachment members 62.

The torsion device 61 may be stretched when certain force is applied to it. For example, the extension device 60 may be situated to apply pressure to one or more points of the torsion device 61 when the extension device 60 is in the first position (shown in FIG. 6A and FIG. 6B). In the first position, the extension device 60 may have a maximal or near-maximal overall length due to minimal or near-minimal longitudinal overlap between the arms 66. The extension device 60 may be sized such that, in the first position, the extension device 60 may be configured to hold the torsion device 61 in a stretched position. In the stretched position, the torsion device 61 may apply minimal or no force to the papillary muscles and/or ventricular walls. For example, the torsion device 61 may be sized and/or configured such that, in the stretched position, the torsion device 61 may have a length approximately equivalent to a distance between papillary muscles and/or ventricular walls.

When the locking mechanisms 64 dissolve and/or are removed, the extension device 60 may no longer be held in the first position. The extension device 60 may be sized such that, when the extension device 60 is in a collapsed position (i.e., when there is maximal or near-maximal longitudinal overlap between the arms 66) the extension device 60 may have a smaller length than the torsion device 61 when the torsion device 61 is in a collapsed position. Accordingly, when the extension device 60 is not held in the first position, the extension device 60 may apply minimal or no force to the torsion device 61. Thus, when the locking mechanisms 64 dissolve or are removed, the torsion device 61 may contract to a collapsed or semi-collapsed position.

By contracting to a collapsed or semi-collapsed position, the torsion device 61 may apply torsional pulling force to one or more papillary muscles and/or ventricular walls, causing the papillary muscles and/or ventricular walls to move closer together. For example, the torsion device 61 may be configured to at least partially pull the posterior-medial papillary muscle 15p and/or the anterolateral papillary muscle 15a towards each other. That is, the torsion device 61 may pull the posterior-medial papillary muscle 15p towards the anterolateral papillary muscle 15a and/or the torsion device 61 may pull the anterolateral papillary muscle 15a towards the posterior-medial papillary muscle 15p, which may cause the papillary muscles to reposition inward. With respect to embodiments in which the extension device 60 and/or torsion device 61 is/are implanted in the right ventricle, the extension device 60 and/or torsion device 61 may serve to correct tricuspid regurgitation.

A delivery system for the extension device 60 and/or torsion device 61 may include a catheter for navigating the extension device 60 and/or torsion device 61 to the desired position. The extension device 60 and torsion device 61 may be delivered separately or together. For example, the extension device 60 may be delivered to the implantation location in the first position and in contact with and/or connected to the torsion device 61. The extension device 60 may adjust to a collapsed position after a period of time. The extension device 60 and/or torsion device 61 may be inserted non-surgically in, for example, a transcatheter procedure (e.g., transfemoral, transseptal, transapical, etc.), wherein the pinch device 50 is inserted into the left ventricle 3 from the aorta 12 through the aortic valve 7 and positioned between the papillary muscles and/or ventricular walls. With respect to right ventricle papillary muscle repositioning, the extension device 60 and/or torsion device 61 may be inserted into the right ventricle from the pulmonary artery through the pulmonary valve and positioned between the papillary muscles of the right ventricle.

### Cardiac Tissue Repositioning Processes

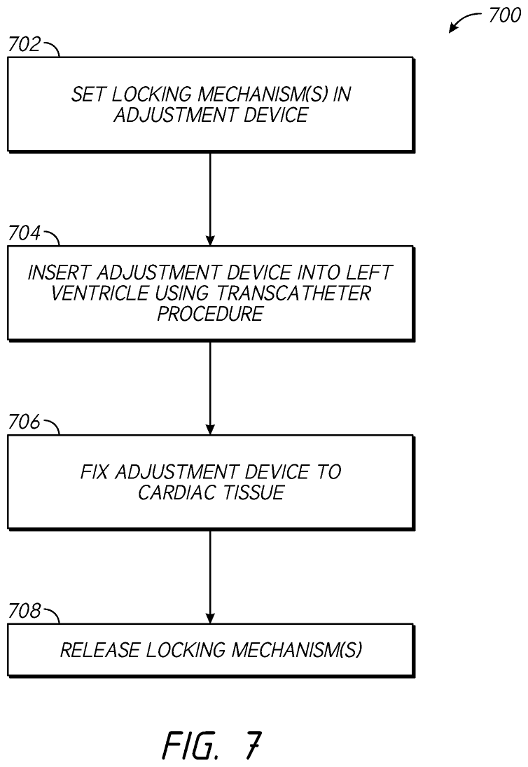

FIG. 7 is a flow diagram representing a process 700 for repositioning ventricular walls, one or more papillary muscles, and/or other anatomy of a ventricle of the heart according to one or more embodiments disclosed herein. While some steps of the process 700 may be directed to the left ventricle, such steps may also be applied to the right ventricle.

At block 702, the process 700 involves setting one or more locking mechanisms in a repositioning device. The repositioning device may be the tension device 40 (FIG. 4), pinch device 50 (FIG. 5), extension device 60 and/or torsion device 61 (FIG. 6), or other device configured to reposition papillary muscles and/or other heart anatomy. The one or more locking mechanisms may be lines or sutures, spacers, and/or other mechanisms configured to hold the repositioning device in a first position. The one or more locking mechanisms may be set in the repositioning device outside or inside a patient's body. In certain embodiments, the one or more locking mechanisms may be connected to one or more portions of the repositioning device. In some embodiments, the one or more locking mechanisms may be inserted into one or more cavities, gaps, apertures, or voids of the repositioning device and/or may be tied around one or more portions of the repositioning device. The one or more locking mechanisms may be composed of a naturally-dissolving material that may dissolve after a period of time while inside a patient's body.

At block 704, the process 700 involves inserting the repositioning device into a ventricle of the heart, such as the left ventricle, using a transcatheter procedure. For example, the repositioning device may be delivered using a transfemoral, transendocardial, transcoronary, transseptal, transapical, or other approach. Alternatively, the repositioning device may be introduced into the desired location during an open-chest surgical procedure, or using other surgical or non-surgical techniques known in the art. In accordance with certain embodiments, the repositioning device may be positioned between two or more papillary muscles of the left (or right) ventricle.

At block 706, the process 700 involves fixing or securing the repositioning device to one or more papillary muscles (e.g., the anterolateral and posterior-medial papillary muscles) and/or ventricular walls. It may be desirable for the repositioning device to be positioned and/or sized such that the repositioning device may apply no force or a minimal amount of force to the papillary muscles and/or ventricular walls at the time of insertion. The repositioning device may be fixed to the ventricle wall with any suitable or desirable anchors or attachment mechanisms.

At block 708, the process 700 involves releasing the one or more locking mechanisms. In certain embodiments, the one or more locking mechanisms may be released after a period of time sufficient for a desired amount of fibrotic tissue to form around the anchors or attachment mechanisms of the repositioning device. For example, the one or more locking mechanisms may be released after a period of several weeks or months. In certain embodiments, the one or more locking mechanisms may dissolve and/or change positions naturally and no removal of the locking mechanisms may be required. For example, the one or more locking mechanisms may be composed of a bio-degradable and/or bio-absorbable material that may be configured to dissolve after a period of time within a patient's body. In such embodiments, the one or more locking mechanisms may be configured to dissolve after a period of time sufficient for a desired amount of fibrotic tissue to form around the anchors or attachment mechanisms of the repositioning device. In some embodiments, the one or more locking mechanisms may be released transcatheter.

The process 700 and/or other processes, devices, and systems disclosed herein may advantageously provide mechanisms for implementing papillary muscle and/or ventricular wall repositioning using a fully transcatheter procedure on a beating heart. In certain embodiments, valve leaflets may not be substantially touched or damaged during the process 700. Furthermore, in certain embodiments, the repositioning device may be designed to be retrievable.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A cardiac device comprising:
a first attachment member configured to be anchored to a first portion of cardiac tissue;
a second attachment member configured to be anchored to a second portion of cardiac tissue;
an adjustable body configured to be moveable between multiple positions; and
a locking mechanism configured to control movement of the adjustable body between the multiple positions.

2. The cardiac device of claim 1, wherein:
the first portion of cardiac tissue comprises a first papillary muscle disposed in a ventricle of a heart, the first papillary muscle being connected to a first leaflet of an atrioventricular heart valve; and
the second portion of cardiac tissue comprises a second papillary muscle disposed in the ventricle of the heart, the second papillary muscle being connected to a second leaflet of the atrioventricular heart valve.

3. The cardiac device of claim 1, wherein:
the first portion of cardiac tissue comprises a first ventricular wall; and
the second portion of cardiac tissue comprises a second ventricular wall.

4. The cardiac device of claim 1, wherein:
the adjustable body is further configured to naturally assume a first position of the multiple positions providing a first distance between the first attachment member and the second attachment member;
the locking mechanism is configured to lock the adjustable body in a second position of the multiple positions for a finite period of time, the second position providing a second distance between the first attachment member and the second attachment member; and
the second distance is greater than the first distance.

5. The cardiac device of claim 1, wherein the locking mechanism is at least partially composed of a naturally-dissolving material.

6. The cardiac device of claim 5, wherein the adjustable body is configured to reposition the first portion of cardiac tissue and the second portion of cardiac tissue after the locking mechanism dissolves.

7. The cardiac device of claim 1, wherein the locking mechanism comprises a spacer disposed between portions of the adjustable body.

8. The cardiac device of claim 1, wherein the locking mechanism comprises a line configured to fit into an aperture in the adjustable body.

9. The cardiac device of claim 1, wherein:
the adjustable body comprises an accordion structure; and
the adjustable body is configured to naturally assume a collapsed configuration of the accordion structure.

10. The cardiac device of claim 1, wherein the adjustable body comprises:
a first elongate arm;
a second elongate arm;
a first connecting arm extending from the first elongate arm; and
a second connecting arm extending from the second elongate arm; and
wherein the locking mechanism is configured to couple to the first connecting arm and the second connecting arm at a connection point.

11. The cardiac device of claim 1, wherein the adjustable body comprises:
a spring; and
a plurality of arm members configured to hold the spring in an at least partially expanded state.

12. The cardiac device of claim 11, wherein:
the plurality of arm members comprises two or more telescoping arms;
one of the two or more telescoping arms is configured to be nestingly fit within another of the two or more telescoping arms; and
the locking mechanism is configured to hold the two or more telescoping arms in an extended position for a finite period of time.

13. The cardiac device of claim 11, wherein the plurality of arm members comprises two or more longitudinally overlapping arms.

14. The cardiac device of claim 1, wherein the first attachment member and the second attachment member are configured to cause formation of fibrotic tissue at the first portion of cardiac tissue and second portion of cardiac tissue, respectively.

15. A method for anchoring into biological tissue, said method comprising:
delivering a cardiac device into a ventricle of a heart using a delivery system comprising a catheter, the cardiac device comprising:
an adjustable body configured to be moveable between multiple positions; and
a locking mechanism configured to control movement of the adjustable body between the multiple positions; and
fixing the cardiac device to a first portion of cardiac tissue and a second portion of cardiac tissue of the ventricle.

16. The method of claim 15, wherein:
the adjustable body further comprises:
a first attachment member configured to be anchored to the first portion of cardiac tissue; and
a second attachment member configured to be anchored to the second portion of cardiac tissue;
the adjustable body is further configured to naturally assume a first position of the multiple positions providing a first distance between the first attachment member and the second attachment member;
the locking mechanism is configured to lock the adjustable body in a second position of the multiple positions for a finite period of time, the second position providing a second distance between the first attachment member and the second attachment member; and
the second distance is greater than the first distance.

17. The method of claim 15, wherein the locking mechanism is at least partially composed of a naturally-dissolving material.

18. The method of claim 17, wherein the cardiac device is configured to reposition the first portion of cardiac tissue and the second portion of cardiac tissue after the locking mechanism dissolves.

19. The method of claim 15, further comprising removing the locking mechanism after fibrotic tissue forms around at least a portion of the cardiac device.

20. The method of claim 15, wherein:
the adjustable body comprises an accordion structure; and
the adjustable body is configured to naturally assume a collapsed configuration of the accordion structure.

21. The method of claim 15, wherein the adjustable body comprises:
a first elongate arm;
a second elongate arm;
a first connecting arm extending from the first elongate arm; and
a second connecting arm extending from the second elongate arm;
wherein the locking mechanism is configured to couple to the first connecting arm and the second connecting arm at a connection point.

22. The method of claim 15, wherein the adjustable body comprises:
a spring; and
a plurality of arm members configured to hold the spring in an at least partially expanded state.

23. The method of claim 22, wherein:
the plurality of arm members comprises two or more telescoping arms;
a first telescoping arm of the two or more telescoping arms is configured to be nestingly fit within a second telescoping arm of the two or more telescoping arms; and
the locking mechanism is configured to hold the two or more telescoping arms in an extended position for a finite period of time.

24. The method of claim 22, wherein the plurality of arm members comprises two or more longitudinally overlapping arms.

25. A cardiac device comprising:
a first means for anchoring to a first portion of cardiac tissue;
a second means for anchoring to a second portion of cardiac tissue;
a tensioning means configured to be moveable between multiple positions; and
a locking means configured to control movement of the tensioning means between the multiple positions.

26. The cardiac device of claim 25, wherein:
the tensioning means is further configured to naturally assume a first position of the multiple positions providing a first distance between the first means for anchoring and the second means for anchoring;
the locking means is configured to lock the tensioning means in a second position of the multiple positions for a finite period of time, the second position providing a second distance between the first means for anchoring and the second means for anchoring; and
the second distance is greater than the first distance.

27. The cardiac device of claim 25, wherein the locking means is at least partially composed of a naturally-dissolving material.

28. The cardiac device of claim 25, wherein the locking means comprises a spacer disposed between portions of the tensioning means.

29. The cardiac device of claim 25, wherein:
the tensioning means comprises an accordion structure; and
the tensioning means is configured to naturally assume a collapsed configuration of the accordion structure.

30. The cardiac device of claim 25, wherein the tensioning means comprises:
a first elongate arm;
a second elongate arm;
a first connecting arm extending from the first elongate arm; and
a second connecting arm extending from the second elongate arm; and
wherein the locking means is configured to couple to the first connecting arm and the second connecting arm at a connection point.

31. The cardiac device of claim 25, wherein the tensioning means comprises:
a spring; and
a plurality of arm members configured to hold the spring in an at least partially expanded state.
